(19) United States
(12) Patent Application Publication
(10) Pub. No.: US 2006/0078512 A1
(43) Pub. Date: Apr. 13, 2006

(54) Liquids to gel

(76) Inventor: Gerald G. McLaughlin, Port Jefferson Station, NY (US)

Correspondence Address: Gerald McLaughlin, 12 Cottonwood Avenue, Port Jefferson Station, NY, 11776, US

(21) Appl. No.: 11/221,321

(22) Filed: Sep. 7, 2005

Related U.S. Application Data
(60) Provisional application No. 60/609,353, filed on Sep. 14, 2004.

(51) Int. Cl. A61K 8/18

(52) U.S. Cl. 424/49

(57) ABSTRACT

A method of forming a gel by combining two or more liquid components at the time of use. An excellent application of such a system would be for tooth whitening, but this system can also be used for other purposes such as hair care, printing inks, cleaning products, and the like. By utilizing this invention it is possible to easily create gels that incorporate components which are generally unstable alone or in combination or in gel form without concern about a short shelf life. This invention has such diverse uses as tooth whitening, concentrated cleaning gels, hair treatment, printing inks, as well as others. A preferred method of packaging and delivery is also disclosed.

### RELATED APPLICATIONS

This application claims priority to and benefit of U.S. Provisional Patent Application No. 60/609353 dated Sep. 14, 2004 titled Liquids to gel, and incorporated by reference herein in its entirety.

### BACKGROUND OF THE INVENTION

This invention relates generally to forming gels at the time of use from combinations that would otherwise be unstable. A preferred embodiment would be useful for the whitening of such structures such as teeth, and more particularly to an improved whitening gel and method for forming and applying such a gel.

One of the most popular dental procedures of recent times is tooth whitening. This is generally achieved through application of an oxidizing agent such as hydrogen peroxide gel or paste to the surface of the teeth. Since there is a direct correlation between the strength of the oxidizing agent used and the rate of whitening, the trend has been to use materials of increasing strength and concentration.

The hard dental tissues are relatively insensitive to damaging effects of strong oxidants but this is not true for the soft tissues. It is therefore critical that the soft dental tissues avoid contact with strong oxidizing materials lest they be burned. Because of this, it is impractical to place strong peroxide solutions directly on the teeth, as liquid tends to drip causing the dentist to lose control of the placement of the material and allowing inadvertent contact between the liquid and the soft tissue. Before the invention of peroxide gels, those few intrepid dentists who attempted tooth whitening tried to use liquid by utilizing a small piece of cotton or other absorbent material to hold a drop of liquid while it was itself carefully held against the tooth. Not only does this procedure require huge amounts of the dentist's time (increasing the expense of such a procedure), but the time requirement of this technique is further exacerbated by the fact that normally only a single tooth can be treated at one time.

It is partly for this reason that gels and pastes containing peroxide are presently the only popular methods being used for rapid tooth whitening. In contrast to a liquid, these materials can be painted on the teeth and left alone because they stay where placed.

There are very few known ways to increase the speed of whitening when using gels. As mentioned already, the most popular is to increase the concentration of the oxidant. Unfortunately, the more concentrated the bleaching material, the less stable it becomes. This presents a large problem for gel manufacturer of concentrations above 5% hydrogen peroxide. Further, high concentrations of hydrogen peroxide or other oxidants attack the structure of gels, thereby gradually making them lose viscosity and gradually changing them into a liquid. Even worse, as the strength of the oxidizers increase, they become increasingly sensitive to any destabilizing factors such as the presence of trace impurities, organic material, metal ions, heat, or alkalinity.

While these destabilizing factors work against any reasonable shelf life, many of these same factors increase the speed of whitening at the time of use. In fact, the presence of some particular destabilizers is generally advantageous to the whitening process, and for this reason they are sometimes added at the time of use. When this is desired for the weaker formulations—those with less than a final concentration equivalent to 10% of hydrogen peroxide or less—the material is supplied as two gels that are mixed at the time of use. The first gel normally holds the oxidizer (such as peroxide), and the second gel contains the components that destabilize the oxidizer (and which also act as a bleaching accelerant).

The most popular delivery system for the two gels consists of a dual barrel syringe with a static mixing tip. It is possible to use a syringe that dispenses unequal amounts of the two gels, but there is not on the market any such syringe that has a ratio of greater than 1:5. Since the highest concentration of even slightly stable hydrogen peroxide gel currently available to dentistry is 35%, it is therefore currently impossible to get a final concentration of the mixed material higher than 29% when mixing two gels from a single syringe.

To get a higher final oxidant concentration, it is presently necessary to use a powder/liquid system. In this system, a liquid containing an oxidizer is added to a powdered gel former such as fumed silicon dioxide and mixed until it forms a paste or gel. In such a system it is possible for the liquid peroxide-containing component to be higher than 35%, thus eliminating one of the constraints of the two gel system. Unfortunately though, a powder/liquid system requires the user to take the time and effort of mixing the powder and liquid in much the same way one would mix up pancake batter. It is for this reason that the powder/liquid systems are not very popular.

There is clearly a need for a system of delivering a highly concentrated multi-component bleaching system that mixes simply and virtually instantaneously and results in a gel or paste that will stay in place when positioned on a tooth.

### SUMMARY OF THE INVENTION

I have invented such a system whereby two or more liquids that would normally be unstable in combination are mixed together at or around the time of use to quickly create a gel, thus tremendously extending the shelf life of the product.

It is also possible to utilize the principles of my invention to dissolve a solid into one or more of the liquids at or around the time of the mixing to achieve the same results.

It should be noted that my invention is not limited to whitening gels, since it is equally applicable to any process that requires the use of a gel where storage would be more advantageous as a liquid. Such a system could be gainfully used for hair coloring, certain inks and paints, palliative or medicinal materials, adhesives, sealants, cleaning agents and other similar products. To simplify the descriptions here, I shall refer to a peroxide whitening gel as representative of the whole class of materials that would benefit by the process whereby two or more stable liquids are combined by the end user at time of use to create a single, possibly unstable gel (or if air is included within the gel, a mousse).

This new system comprises two or more components, at least two of which are in liquid phase. One component includes some active element such as bleaching agent (hydrogen peroxide for example), another component includes a liquefied gelling agent, gel former, or gel precursor that is not in a gel state until acted upon by some other component to convert it into a gel (the acrylic copolymers in Structure 2001 by National Starch, for example, are in liquid form when at an acidic pH and a gel when the pH is raised above about 7.0). Gel formers and gelling agents are sometimes referred to in the marketplace as rheology modifiers. There is also a third component needed which acts as a trigger to inaugurate the change from liquid to gel (which I will refer to here as the “gel triggering agent”). The gel triggering agent is kept apart from the liquefied gelling agent until formation of the gel is desirable.

A preferred embodiment of this invention is the creation of a tooth whitening gel from two liquids. The first liquid includes a gelling agent with a rheologic potential that ranges from liquid to viscous gel, and whose viscosity is sensitive to pH. If this gelling agent is in liquid state at low pH and gels at high pH, then it can be combined with any active component with a pH below 7.0 while remaining in a liquid state. In this example, the second liquid would consist of any combination of ingredients that results in a liquid with a pH above 7.0. When mixed together in the right proportions, the combination of the two liquids results in a final mixture with a pH above 7.0 which causes the entire mixture to thicken into a gel state.

These are not the only possible circumstances to use this invention, however. For example, it is also possible to do essentially the opposite and use a liquefied gelling agent that is in liquid state when the pH is alkaline and use an acidic liquid as the triggering agent. Examples of this second preferred embodiment and others are given below.

While many additional substances beyond those listed here can be used in this invention it is usually possible to combine some or even many of the components together to minimize the total number of liquids required, although it is not a requirement of the invention. Some of the substances incorporated into the final mixture need not even be soluble, as they can be suspended in one of the liquids.

It is also possible to have one or more of the ingredients in a non-liquefied state at the time of mixing. For example, a powder could additionally be combined or mixed into one of the liquid components around the time of use.

One of the features of this invention is that it is possible to leave one or more active ingredients in a non-gelled state until time of use, thus greatly extending their shelf life. And since the components to be mixed at the time of use are generally all in liquid form or dissolvable or otherwise carried by a liquid, the mixing phase can be as simple as giving the container holding the combined ingredients a shake. Sometimes even this little bit of effort is more than necessary, as the liquids often tend to self disperse into each other. It is noteworthy that there can be a wide variation in the ratio between the components.

The result of using this invention is an active material with a long shelf life, but which can quickly be converted into a gel, despite the fact that it can contain extremely strong oxidizing agents and/or destabilizers.

### BRIEF DESCRIPTION OF THE FIGURES

FIG. 1 is a side elevational view of an inverted blister pack containing a liquid.

FIG. 2 is a view of the end user utilizing a syringe containing one of the liquids to pierce into the blister pack shown in FIG. 1.

### DETAILED DESCRIPTION OF THE PREFERRED EMBODIMENTS

In accordance with the principles of this invention, one preferred embodiment is a positionable gel with high peroxide concentration suitable for use in whitening teeth in a clinical setting.

In this embodiment, two liquids are provided to the end user. The first liquid contains highly concentrated hydrogen peroxide mixed together with a small amount of gel former. One appropriate peroxide is Peralkyli, from Degussa, which is available up to 50% concentration, although any stable peroxide can be used. The gel former is specifically chosen to be one which is not affected by the presence of the oxidizer while in liquid state, and which also does not destabilize the peroxide. An appropriate liquefied gel former is Structure 2001 from National Starch, although there are many others available. This gel former remains in liquid form while acidic, but converts to a gel when the pH rises to 7.0 or above. Examples of other suitable gel formers include Acrylates/Ceteth-20 Itaconate Copolymer, Acrylates/Aminoacrylates/C10-30 Alkyl PEG-20 Itaconate Copolymer, carbomers, crosslinked copolymers of acrylic acid and a hydrophobic comonomer, copolymers of polyethylene oxide and polypropylene oxide. The Peralkyli has a low pH, and so the mixture of these two liquids results in a stable milky liquid. Because of the combination of stabilizers in Peralkyli, this mixture has been shown in tests to be stable at room temperature for much longer than 12 months. The liquid can be delivered to the end user in any suitable container, but a preferred embodiment provides this liquid to the user in a partially filled hypodermic syringe. With reference to FIG. 2, there is shown an end user holding a hypodermic syringe 3 partially filled with the first liquid 6.

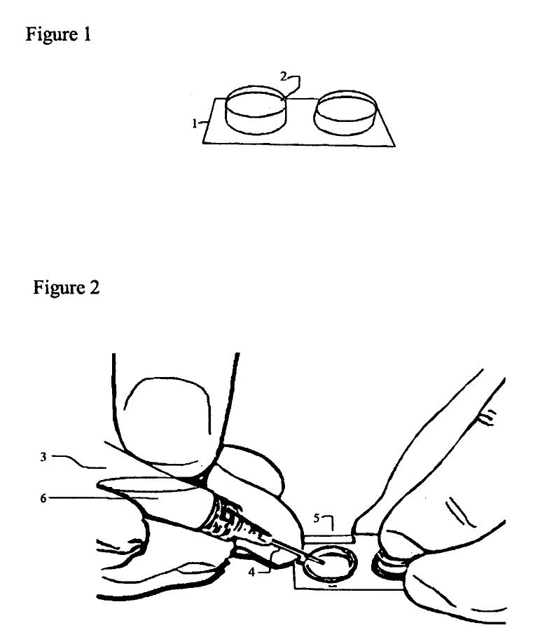

The second liquid in this embodiment uses a concentrated solution of water and sodium hydroxide as the gel triggering agent, although any solution of high pH such as potassium hydroxide, tripolyphosphate, tris(hydroxymethyl)aminomethane, sodium carbonate, ammonium hydroxide, alkylamine or amino alcohol. This second liquid is provided in a container that allows the liquid to be drawn into the syringe by pulling back on the syringe plunger. A preferred packaging of this second liquid is inside a blister pack such as is used to deliver tablets of various sorts, although any suitable container will work including bottles and vials. FIG. 1 shows the blister pack 1 partially filled with a liquid 2. In the case of this preferred embodiment, the sodium hydroxide is sufficiently concentrated to allow only a few drops of the liquid to neutralize and raise the pH of the first liquid as delivered. In this case around seven drops of the second liquid are placed into the well of a blister pack, and it is sealed using some inert covering such as polyethylene. Mixed into the second liquid can be various bleaching accelerators including those chosen from the groups of metal ions (such as incorporated through magnesium gluconate, manganous chloride, manganese sulfate monohydrate, or ferrous sulfate), coloring agents such that would respond to specific wavelengths of light, surfactants, amino acids, peroxide converters, and/or an enzyme such as catalase, peroxidase, dopamine β-hydroxylase, and peptide glycine α-hydroxylase.

In use, the end user simply uses the hypodermic syringe tip shown in FIG. 2 as 4 to perforate through the covering of the blister pack 5 and rapidly sucks the second liquid into the syringe, causing the two liquids to mix. Within a short period of time (sometimes as short as fifteen seconds) the two liquids combine to form a clear or translucent gel that can be applied to the teeth using the same syringe as a dispenser. To speed up the mixing and gelling of the liquids, the syringe can be gently shaken.

If the manufacturer prefers to leave the decision as to the final concentration of the peroxide in above example whitening gel, it is possible to use three liquids. In this case the gel forming agent could be delivered in a syringe, and the peroxides of various strengths could be available in a separate vial. In such a case, the first step of the end user would be to draw the peroxide into the syringe, thus mixing the peroxide and the gel former. Once so mixed, the end user would then draw the gel triggering agent into the syringe as described above.

### NON-LIMITING EXAMPLES

### Example 1

In one embodiment of this invention, an acrylic copolymer such as Structure 3001 (National Starch) is used as the gelling agent. It is in liquid form when the pH is below around 7, but the copolymer swells to form a clear gel when the pH is raised above 7. The polymer is compatible with strong hydrogen peroxide, so one component of the system is a liquid containing 35% hydrogen peroxide and a small amount of Structure 3001. The second component consists of water and a pH adjuster such as sodium hydroxide. The pH adjuster could be (as in this case) any soluble alkaline material (including, for example, tris amino, AMPD, potassium hydroxide, sodium hydroxide, AMP, ammonium hydroxide, sodium percarbonate, etc). In addition, there could be other materials added for their particular properties, such as glycerin, propylene glycol, pigments, aloe vera, magnesium gluconate, emollients, stabilizers, destabilizers, flavors, sweeteners, fragrances, tooth hardeners such as fluoride, and other treatment materials and esthetic enhancers. In this example using Structure 3001 and hydrogen peroxide, the first liquid appears milky white, and the second appears clear or cloudy depending upon what materials are suspended or dissolved or suspended. The most important characteristic of the second material is that the pH must be high enough that when the selected amount of this liquid is added to the first liquid component, the pH of the resulting mixture is above 7. Then when mixed together and agitated well, the combination of the first and second components almost instantly become a clear fully-activated gel complete with whitening accelerators and ready for placement.

### Example 2

In another example, I mixed sodium percarbonate and carbamide peroxide with glycerin as the oxidizer and pH modifier. My second liquid component consisted of Structure 3001 plus water, fragrance, and flavoring. When these were mixed, an activated, destabilized oxidative bleaching gel resulted.

### Example 3

In yet another example, my oxidizer and pH modifier was Oxone in a glycerin base. The gelling agent was Structure Plus (National Starch) which is liquid when the pH is alkaline and gelled when acidic and water. At the time of use, the two liquids were combined, and a positionable non-peroxide bleaching gel was the result.

### Example 4

I mixed up a combination of 35% hydrogen peroxide with a small amount of carbomer (Carbopol Ultrez 2 from Neodol, Inc.) to make a slightly thickened liquid with a pH of approximately 4.0 as the first component. I then made a mixture of water, sodium hydroxide, a red dye, and silver nitrite as the second component. Upon mixing these two materials, an activated gel was formed which included components sensitive to the addition of light energy. When this gel was placed on teeth and a strong light was directed at the gel, the peroxide was further destabilized and bleaching was further accelerated.

### Example 5

For this example, I took the first component in Example 1 and placed 1.5 ml into a 3 ml syringe. I also made a second chamber in the syringe using polyethylene film as the divider. This chamber was filled with eight drops of the second component and the plunger was gently inserted into the syringe so as to not break the barrier between chambers. When the syringe was forcefully depressed, the polyethylene film separating the two chambers burst and the liquids mixed. Within five seconds the liquids had changed to a gel. It was then placed on teeth and after about two more minutes the gel began to foam indicating that the peroxide was breaking down releasing free radicals and other active oxidants.

### Example 6

For this example, I took a 3 ml syringe and divided the barrel into two unequal-sized chambers using a soft rubber stopper similar to that found on the end of the syringe plunger. This divider included a one way valve which allowed the contents from the chamber closest to the plunger (the second chamber) to pass through into the first chamber, but would not allow movement in the opposite direction. The first chamber contained 1.5 cc of the first component in Example 1, and the second chamber contained eight drops of the second component. When the plunger was depressed, the second component passed through the valve into the first chamber and the liquids mixed. Within five seconds the liquids had changed to a gel. Further depression of the plunger caused the plunger to come into contact with the barrier and the barrier then joined with the plunger to express the newly formed gel onto the teeth.

### Example 7

In this example I made a mixture of urea peroxide and glycerin adjusted to a pH of 5.5 using sodium hydroxide and containing Dequest as a stabilizer. As a second component I used Aculyn 22 (ISP Technologies, Inc.). As a third component, I used a 50% solution of sodium hydroxide. Using a blunt needle in a 3 cc syringe, I drew 1.5 cc of the first component into the syringe. I then drew 0.3 cc of the second component and shook the syringe. Finally, I drew in 0.3 cc of the third component and shook the syringe again. After about four seconds the liquids had turned into a fast acting, accelerated, positionable gel for whitening teeth.

### Example 8

In this example I placed 0.8 cc of 40% hydrogen peroxide liquid into a 1 cc Luer Lock syringe along with several drops of Structure 2001 (National Starch). At the time of use, this syringe was fitted with a blunt-tipped needle whose plastic hub was packed with granular sodium hydroxide. The grains of the sodium hydroxide were too large to readily pass through the lumen of the needle. As the peroxide and Structure 2001 mixture passed over the sodium hydroxide, some of the sodium hydroxide was dissolved in the liquid, causing the liquid that was being expresses to instantly change to a positionable gel.

Other examples using mixtures of sodium hydroxide and other pH regulators with other soluble components (such as metal ligands, potassium hydroxide, or colored dyes) and even some insoluble components (such as fumed silicon dioxide which are added for such purposes as thickener, accelerator, tissue conditioner, or desensitizer) were tested and found to successfully form useable gels.

In all of the cases illustrated above the resulting gel can be placed on the teeth or any other object where bleaching or oxidation or treatment with a gel is desired. If the object is oxidation, and if the operator so chooses, the oxidative action can be further accelerated through the use of a source of light, heat, or other energy application or transference directed at the gel so produced. If light acceleration is anticipated, then photosensitive agents are often also incorporated into the gel mixture.

While only certain preferred embodiments of this invention have been described; those skilled in the art will appreciate the number of variations and changes that can be made in the preferred methods hereof without departing from the spirit and scope of the invention. Such changes may include differences in specific method steps and/or materials, so long as the essentials of the invention are satisfied. My disclosure is, of course, intended to cover all such variations and changes within the ambit of the invention and not limited by the examples described above.

What is claimed is:

1. A method of creating at or near the time of use a gel comprising from two or more liquids.

2. The method of using a gel formed at or near the time of use from two or more liquids.

3. A gel created at or near the time of use from two or more liquids.
